(19) United States
(12) Patent Application Publication
(10) Pub. No.: US 2010/0291214 A1
(43) Pub. Date: Nov. 18, 2010

(54) THREE-DIMENSIONAL MICROFIBER EXTRUDATE STRUCTURE AND PROCESS FOR FORMING THREE-DIMENSIONAL MICROFIBER EXTRUDATE STRUCTURE

(75) Inventors: Peter D. GABRIELE, York, PA (US); Andrew HOGAN, Red Lion, PA (US)

Correspondence Address: MCNEES WALLACE & NURICK LLC, 100 PINE STREET, P.O. BOX 1166, HARRISBURG, PA, 17108-1166, US

(73) Assignee: ARMARK AUTHENTICATION TECHNOLOGIES, LLC, York, PA (US)

(21) Appl. No.: 12/823,592

(22) Filed: Jun. 25, 2010

Related U.S. Application Data
(63) Continuation-in-part of application No. 12/342,830, filed on Dec. 23, 2008.
(60) Provisional application No. 61/220,770, filed on Jun. 26, 2009.

(51) Int. Cl. A61K 9/00; B05D 3/00

(52) U.S. Cl. 424/486; 427/2.1

(57) ABSTRACT

Disclosed is a three-dimensional microfiber extrudate structure and a process of forming a three-dimensional microfiber extrudate structure. The three-dimensional microfiber extrudate structure includes a matrix having a three-dimensional geometry wherein the three-dimensional geometry is a visco-elastic relaxation state of a preform introduced to a medium.

### PRIORITY

This application claims priority and benefit of U.S. patent application Ser. No. 12/342,830, filed Dec. 23, 2008, and U.S. Provisional Patent Application No. 61/220,770, filed Jun. 26, 2009, both of which are hereby incorporated by reference in their entirety.

### FIELD

The present invention relates to three-dimensional extrudate structures and methods of forming three-dimensional extrudate structures. More specifically, the present invention relates to three-dimensional extrudate structures capable of delivering active loads such as therapeutic loads or diagnostic loads.

### BACKGROUND

Microsphere or polymer microparticle drug delivery manufacturing can include oil in water or water in oil emulsion methods which may limit drug inclusion to those drugs which are soluble in one of the intended phases. The solvents and surfactants necessary to achieve emulsions can represent significant hurdles in obtaining regulatory approval.

Drug delivery devices can be subject to the time and cost associated with FDA compliance prior to being used for cancer diagnostics and therapies. FDA compliance requires that new drugs and devices meet certain regulatory requirements prior to being utilized in the market. For drugs, new drug applications can be full new drug applications, abbreviated new drug applications, or an application that contains full reports of investigations of safety and effectiveness but where at least some of the information required for approval comes from studies not conducted by or for the applicant and for which the applicant has not obtained the right of reference. This third type of new drug application (from section 505(b)(2) of the Food, Drug, and Cosmetic Act) can be quicker and less costly than other types of new drug applications.

R. Krishnamoorti, “Pathway and Kinetics of Cylinder-to-Sphere Order-Order Transition in Block Copolymers,” published Mar. 6^{th}, 2000, in Macromolecules (hereinafter, Krishnamoorti), which is incorporated by reference in its entirety, discusses formation of certain polymer based microstructures. Krishnamoorti suggests that a process including application of large-amplitude shear converts cylinders into spheres. This process suffers from the drawback that it reverses and converts the spheres back into cylinders. Furthermore, the microstructures may not be safe for humans.

Other microstructures suffer from the drawback that they build up within certain areas of the human body. For example, microstructures can build up in the lungs forming an embolism.

Furthermore, pharmaceutical companies continue to come up with new drugs that are often rather insoluble in many common solvents. Particularly, these new drugs are insoluble in water and therefore have limited usefulness in traditional drug delivery methods. To utilize these new developments, a new delivery method for drugs is desirable.

What is needed is a microstructure and a method of forming a microstructure, wherein the microstructure is capable of maintaining a three-dimensional geometry, consists essentially of materials that are safe for humans, and/or is capable of deforming to avoid accumulation within certain areas of the human body.

### SUMMARY

In one aspect of the present disclosure, a process for forming a three-dimensional microfiber extrudate structure includes introducing a preform to a medium and maintaining the preform in the medium at least until a visco-elastic relaxation state is reached. In this aspect, the three-dimensional microfiber extrudate structure is formed by the preform reaching the visco-elastic relaxation state.

In another aspect of the present disclosure, a three-dimensional microfiber extrudate structure includes a matrix having a three-dimensional geometry. In this aspect the three-dimensional geometry is a visco-elastic relaxation state of a preform introduced to a medium and the three-dimensional geometry is deformable.

In another aspect of the present disclosure, a three-dimensional microfiber extrudate structure includes a matrix having a three-dimensional geometry. In this aspect, the three-dimensional geometry is a visco-elastic relaxation state of a preform introduced to a medium and the matrix consists essentially of materials that are safe for humans.

An advantage of the present disclosure includes being capable of maintaining a three-dimensional geometry of a microfiber extrudate structure after drying the microfiber structure.

Another advantage of the present disclosure includes being capable of delivering active loads that are insoluble in water.

Another advantage of the present disclosure includes being capable of combining loads that are otherwise incompatible.

Another advantage of the present disclosure includes being safe for humans.

Another advantage of the present disclosure includes being capable of deforming to avoid accumulation within certain areas of the human body.

Features and advantages of the present disclosure will be apparent from the following more detailed description of the preferred embodiment, taken in conjunction with the accompanying drawings which illustrate, by way of example, the principles of the disclosure.

### BRIEF DESCRIPTION OF THE FIGURES

FIG. 1 shows an electron micrograph of an exemplary preform according to the disclosure.

FIG. 2 shows a schematic view of an exemplary preform according to the disclosure.

FIG. 3 shows a cross-section of an exemplary three-dimensional microfiber extrudate structure according to an embodiment of the disclosure.

FIG. 4 shows an exemplary micro-extruder.

FIG. 5 shows an electron micrograph of an exemplary preform according to the disclosure.

FIG. 6 shows an electron micrograph of an exemplary three-dimensional microfiber extrudate structure according to an embodiment of the disclosure.

FIG. 7 shows an electron micrograph of an exemplary three-dimensional microfiber extrudate structure according to an embodiment of the disclosure.

FIG. 8 shows a cross-section of an exemplary three-dimensional microfiber extrudate structure according to an embodiment of the disclosure.

FIG. 9 shows a cross-section of an exemplary three-dimensional microfiber extrudate structure according to an embodiment of the disclosure.

FIG. 10 shows a characteristic particle size distribution of a collection of an exemplary three-dimensional microfiber extrudate structure produced in accordance with an exemplary embodiment of the disclosure.

FIG. 11 shows a process according to an exemplary embodiment of the disclosure.

FIG. 12 shows another process according to an exemplary embodiment of the disclosure.

### DETAILED DESCRIPTION

Provided is a method of making a three-dimensional microfiber extrudate structure and a three-dimensional microfiber extrudate structure capable of delivering active loads such as therapeutic loads or diagnostic loads.

Embodiments of the present disclosure can be capable of maintaining a three-dimensional geometry of a three-dimensional microfiber extrudate structure after drying the three-dimensional microfiber extrudate structure, can be capable of delivering active loads that are insoluble in water, can be safe for humans, and/or can deform to avoid accumulation within certain areas of the human body such as the circulatory system of the lungs.

One embodiment of the disclosure is a microfiber extrudate including up to 100% solid, stable, multifunctional, polymeric microcarrier delivery vehicles for active pharmaceutical ingredients (API), nanoparticles and/or additional bioagents using 100% FDA compliant components.

The three-dimensional microfiber extrudate structure can provide a vector carrier for intravenous, intra-peritoneal, parenteral, oral, gastric, colonic, lavage, or superficial targeted delivery application. The term “microfiber extrudate” as used herein includes microvectors, microcells, microspheres, artificial cells, nano-particles and other suitable devices. The three-dimensional microfiber extrudate structure can be consistent with a stable-core-constituent multicomponent targeting strategy in nanobiotechnology delivery and/or can be a nanocarrier. The three-dimensional microfiber extrudate structure can include geometric forms such as ellipses, cylinders, microneedles, and nanofibers for tissue engineering. The design of the three-dimensional microfiber extrudate structure can be spatially resolvable, which permits a deliberate placement of active and passive components within the three-dimensional microfiber extrudate structure, as will be discussed in more detail herein. Feature size and shape can be controlled, which may permit creation of the three-dimensional microfiber extrudate structure in actual sizes and geometry that correspond to desired sizes and geometries. The predetermined size and geometry may be intended to mimic the size of a cell. For example, the three-dimensional microfiber extrudate structure may be configured to have a size and geometry similar to a red blood cell or a white blood cell for a specific animal (including humans).

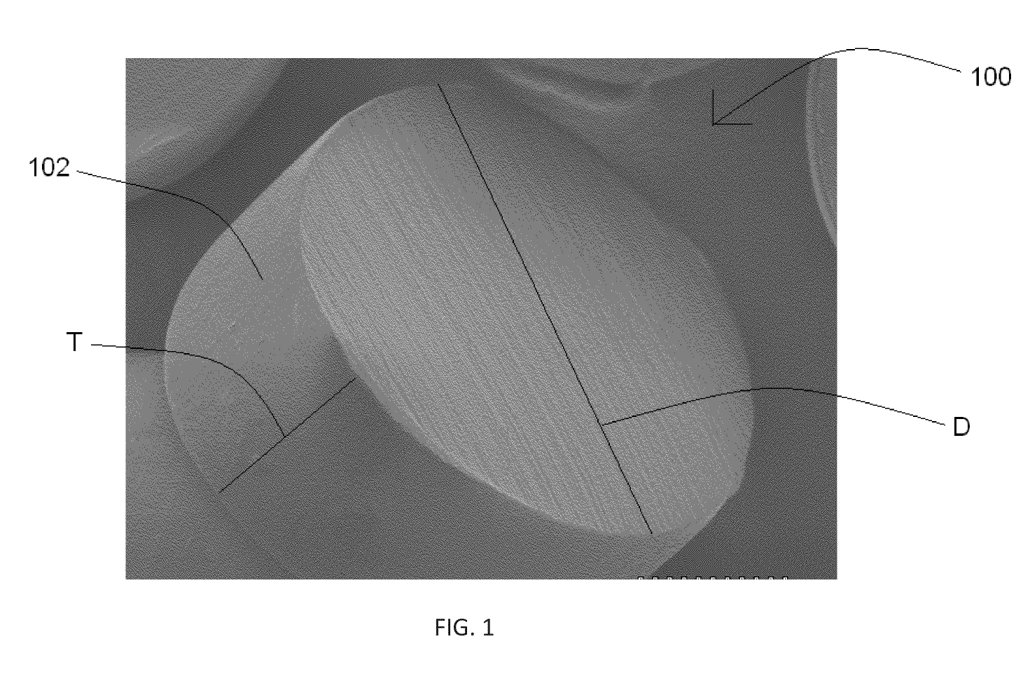

Referring to FIG. 1, in one embodiment, a microfiber extrudate or preform 100 includes a matrix, an exogenously excitable material, and an active load. The matrix may include a radiosensitive active pharmaceutical drug, an antibody, a chemotherapeutic agent, neat copolymer, an API, thermoplastic material that is biologically compatible, vascular-infusible and bio-compatible material, and/or other suitable material as will described herein. The matrix forms a body 102 of the preform. The body 102 defines the exterior of the preform 100. The body 102 may be (but is not necessarily) circular in cross-section and may be designed to have a predetermined diameter (for example, about 5 μm to about 10 μm or to about 300 μm or larger). In one embodiment, the body 102 includes a diameter D of about 100 μm. The body 102 may have a transverse thickness (for example, as small as about 5 μm) In one embodiment, the body 102 has a transverse thickness T of about 10 micrometers. The body 102 may be elongate or spherical.

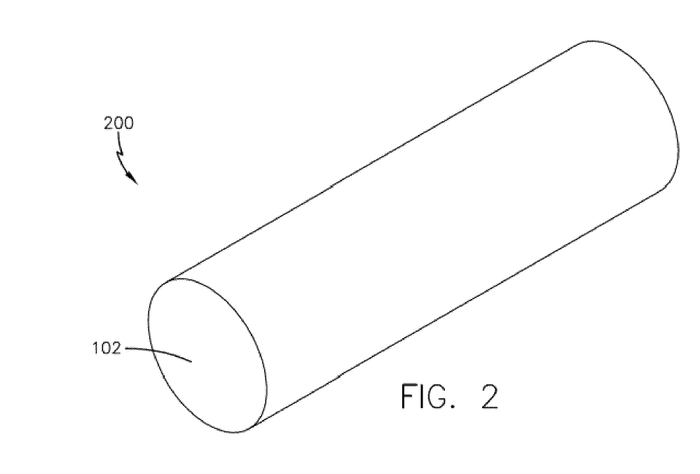

FIG. 2 shows an exemplary embodiment of a microfiber extrudate or preform 200. Here, the preform 200 is elongate. The preform 200 may be transversely sliced along its cross-section to make a plurality of axial slices substantially the same as the preform 100 shown in FIG. 1.

The preform 100 can be manufactured from any extrudable polymeric composition that is safe for humans including, for example, FDA compliant polylactic-glycolic acid co-polymer extrudate (PLGA), or similar FDA compliant biodegradable polymer that has been commingled with an active agent of biological interest. The polymer extrudate composition can be initially pre-processed into a master batch including the API, a nano-agent, and/or similar combination of components including imaging agents before being arranged into a micro-rod or micro-fiber.

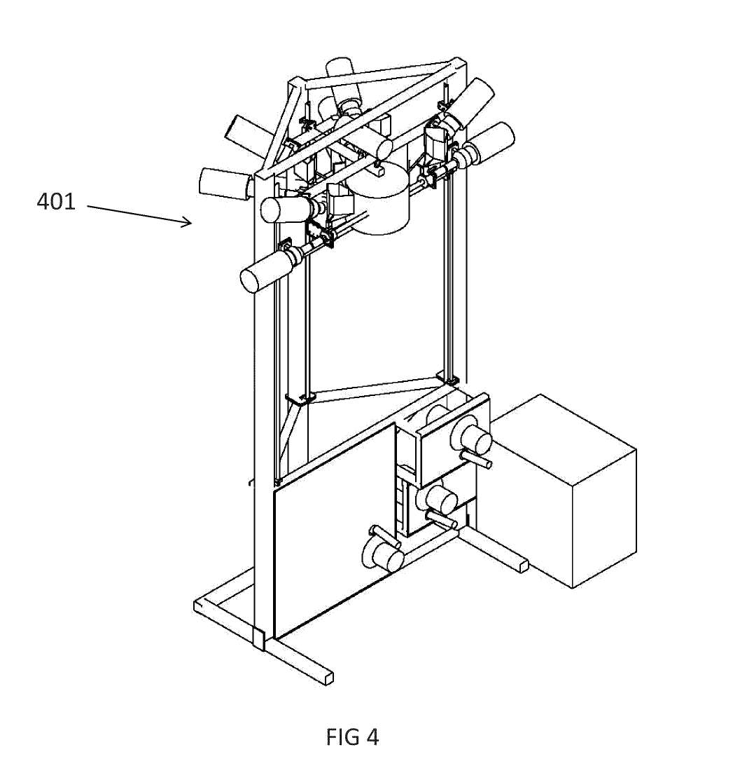

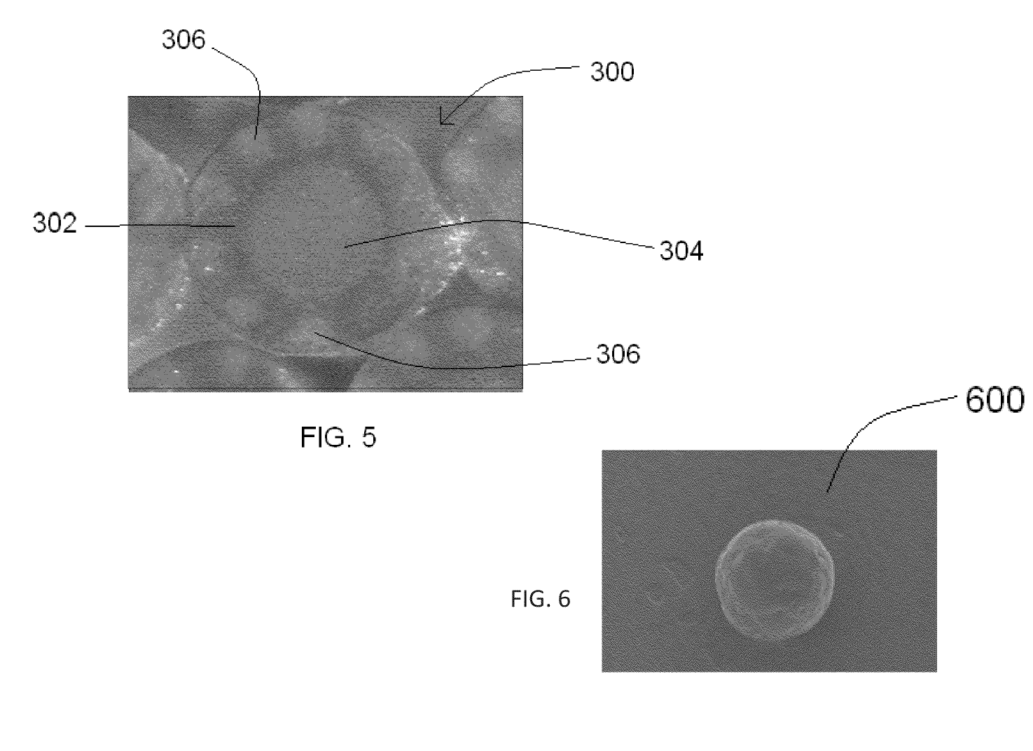

Referring to FIG. 4, a process of forming the preform can be a high definition micro-extrusion process. The process can utilize several extruder barrels that intersect into a specially designed “die head,” such as disclosed in WO 2007/134192. Each barrel delivers a single component for subsequent combination within the die head. The die head is configured such that the matrix, the exogenously excitable materials, and the active load exiting the multiple extruder barrels enter a series of pixilated stacked die plates, called a die-pack. A unique die-pack may be provided for each different preform design. The total pixel bundle exiting the last plate may contain up to 21,000 or more nano-fibers, which coalesce at the spin head into a single fiber. Additionally or alternatively, other suitable processes (for example, using a die face cutter) may be used.

In one embodiment, the formation/construction of the preform may be performed using a micro-extrusion fiber spinning process. In this process, a precision engineered die can define intended domains as nano-fiber regions that, when combined at the spinning head, anneal into one single fiber having any number of deliberately defined internal domains. This produces a so-called “island-in-the-sea” arrangement of one or more different materials (e.g., active loads and/or exogenously excitable material) as “islands” within the matrix or “sea” of a base material. Suitable devices and methods for co-extruding a filament of different components in a pre-determined spatial arrangement are described, for example, in U.S. Pat. Nos. 4,640,035; 5,162,074; 5,344,297; 5,466,410; 5,562,930; 5,551,588; and 6,861,142 and in WO 2007/134192, all of which are herein incorporated by reference.

These processes allow the co-fabrication of several material components within the “design space” of the three-dimensional microfiber extrudate structure. The three-dimensional microfiber extrudate structure can include three to four material components; more or fewer may be incorporated. The material components can be spatially resolved and freely positioned by design within the body of the three-dimensional microfiber extrudate structure. It will be appreciated that the three-dimensional microfiber extrudate structure may be created by co-extruding pure materials for the matrix and each domain, or the components of the three-dimensional microfiber extrudate structure may themselves be a mixture of material(s) with the desired properties (for example, the properties of the exogenously excitable materials and/or the active load) arranged in discrete domains or as the matrix, which may assist in the coextrusion of the materials.

The three-dimensional microfiber extrudate structure can be solid, stable, and generally are not hollow. The three-dimensional microfiber extrudate structure containing solutions such as those prepared by emulsion technologies like liposomes. Furthermore, compatibility is not limited to solubility with exemplary embodiments because the process can include solid-solid dispersion at selected melt-flow temperatures. Thus, an insoluble API can be commingled within the polymer matrix. The three-dimensional microfiber extrudate structures can include a polymeric matrix from the surface all the way to the inner core, with the API incorporated into the polymer matrix. Therefore, complex solution and surface agent chemistries required for emulsion preparation can be avoided. Furthermore, drug solubility can be of less or no concern to the processing, allowing API with difficult to manage solubility properties to be incorporated as easily as API that are readily soluble. This process can also allow for the inclusion of multiple API with very different solubility parameters, as they are stabilized in the polymer matrix in the master-batch process and simultaneously delivered upon degradation of the matrix. In one embodiment, the matrix includes at least two otherwise incompatible components. As used herein, the term “otherwise incompatible components” refers to components that traditionally could not be combined in drugs. For example, components insoluble in combination are otherwise incompatible. In addition, components that react upon contact to each other are otherwise incompatible. In another embodiment, the matrix includes a crystalline drug dispersed in a polymer thereby forming an amorphous drug.

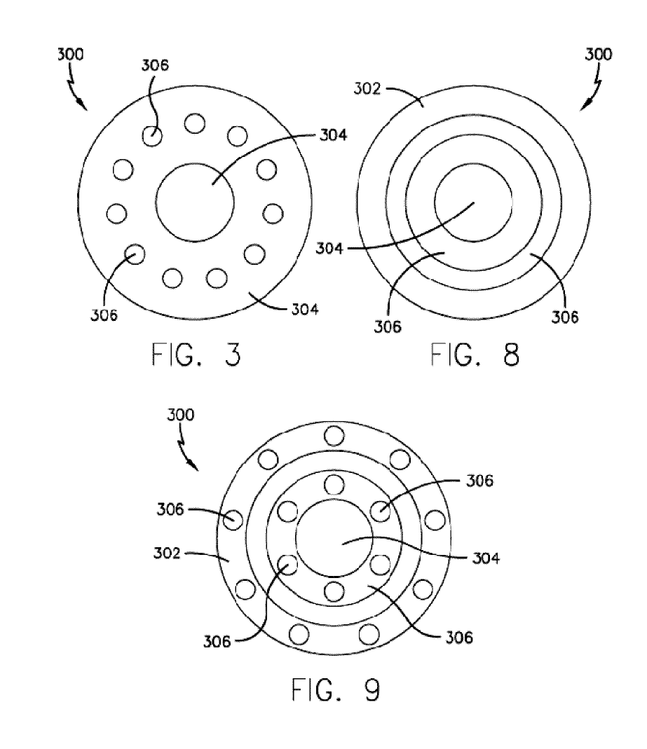

In one embodiment, the three-dimensional microfiber extrudate structure may include a plurality of discrete domains, such as shown in FIG. 3, and can undergo further processing before visco-elastic transformation to yield further advantages. For example, the three-dimensional microfiber extrudate structure may be subjected to a treatment in a solvent in which the composition of the matrix, but not the discrete domains are soluble. This can effectively result in the removal of the matrix and thus the separation of the discrete domains into independent particles that can then be individually subjected to the visco-elastic transformation processes described herein to create even smaller individual particles. This may be used, for example, to form nano-particles of neat API for subsequent use.

A general extrusion process according to the disclosure includes forming the three-dimensional microfiber extrudate structure by producing the preform (for example, a preform micro-rod or micro-fiber), further processing the preform (for example, into coin-like cylinders), and introducing the preform to a medium to form the three-dimensional microfiber extrudate structure (for example, microspheres).

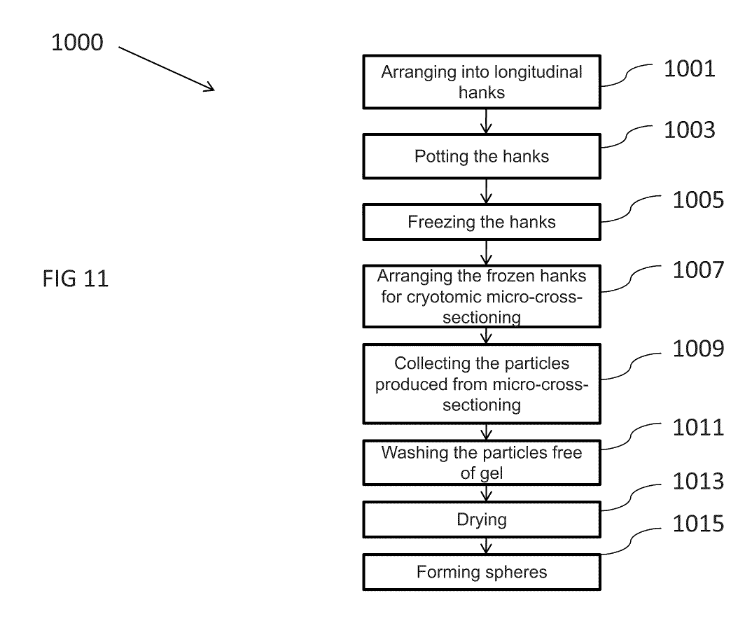

Referring to FIG. 11, an exemplary extrusion process (process 1000) includes arranging the preform en mass in longitudinal hanks (step 1001). The hanks can be potted into sectioned blocks in a thixotropic potting gel, such as FDA compliant aqueous 2% solids dispersible cellulosic thickening agent (step 1003). The hanks can then be frozen (step 1005). In one embodiment, the hanks are frozen at a predetermined temperature (for example, about −23° C.) and for a predetermined period (for example, about 24 hours). The frozen blocks can then be arranged for cryotomic micro-cross-sectioning (step 1007). For example, a blade can be positioned perpendicular to the hanks. The blade can produce “coin-like” cylinder particles as the microfiber extrudate. In one embodiment, the preform can have a predetermined aspect ratio (for example, between about 5:1 and 7:1, diameter to height). In addition to coin-like cylinders, it will be understood that the preform may include any predetermined shape (for example, square slices, oval slices, cubes, etc.) capable of being formed based upon the cross-sectional shape of the fiber and the manner in which the fibers are sectioned. The preform can be collected (step 1009) and washed free (step 1011) of the gel. Then, the preform can be dried (step 1013). In one embodiment, the preform can be dried at about 60° C. for about 20 minutes under continuous air flow.

In one embodiment, the three-dimensional microfiber extrudate structure may be formed by phase-exclusion viscoelastic thermal-sphericalization (PEVTS) in a confined reservoir of an FDA compliant medium such as soybean oil followed by a pharmaco-compliant “detergent” wash to render the final three-dimensional microfiber extrudate structure. The preform can be treated to transform its shape and/or geometry into any suitable shape and/or geometry. The change in shape and/or geometry can include producing a biomimetic delivery system in the natural range of circulatory cells, transforming the entire shape and/or geometry of the three-dimensional microfiber extrudate structure (for example, transforming the matrix of the microfiber extrudate), and/or transforming the shape and/or geometry of a portion of the three-dimensional microfiber extrudate structure (for example, transforming the domains in the matrix of the three-dimensional microfiber extrudate structure). For example, the preform can be a coin-like cylinder particle capable of being formed into the three-dimensional microfiber extrudate structure that can be spherical (step 1015). It will be appreciated that the transformation may not result in a perfect sphere, and that the ultimate geometry of transformation may be rod-like or of any other geometry relative to the shape of the extrudate prior to the transformation process.

In another embodiment, the preform 100 as shown in FIG. 1 can be transformed to a sphere 600 shown in FIG. 6 by placement in a suitable medium (for example, a 50% ethanol and 50% water solution). The components and relative percentages of which may be adjusted based upon the particular polymer and API combination. For example, poly ethylene glycol (PEG) can be used to swell the extrudate 100. The matrix of the preform 100 can be configured to have increased osmotic potential and may include hypertonic materials, for example, salt, that permit the microfiber extrudate to transform or swell under selected conditions. The transformation into the sphere 600 may increase the efficacy of a thermally-sensitive active pharmaceutical ingredient. Unexpectedly, the three-dimensional microfiber extrudate structure can generally maintain its three-dimensional (for example, sphere-like) geometry after being dried.

The process of converting the preform into the three-dimensional microfiber extrudate structure can permit the three-dimensional microfiber extrudate structure to incorporate other materials introduced after extrusion. For example, PEG can be used for performing “PEGylation” that brings a suitable material, for example a nano-particle, into the three-dimensional microfiber extrudate structure. To increase the ability to incorporate other material into the three-dimensional microfiber extrudate structure, the geometry of the three-dimensional microfiber extrudate structure may be configured to provide increased surface area and/or decreased surface area. This may be achieved by modifying the extrusion process or by modifying the three-dimensional microfiber extrudate structure after it is extruded. In an exemplary embodiment, PEGylation can be used for bringing binding agents, such as macrophages, into the three-dimensional microfiber extrudate structure or the preform, thereby permitting the binding agents to be released through diffusion and/or degradation of the matrix. In another exemplary embodiment, PEGylation can be used for bringing in material, structures, or nano-particles that prevent white blood cells from attacking the three-dimensional microfiber extrudate structure. Additionally or alternatively, the three-dimensional microfiber extrudate structure may be aerosolized.

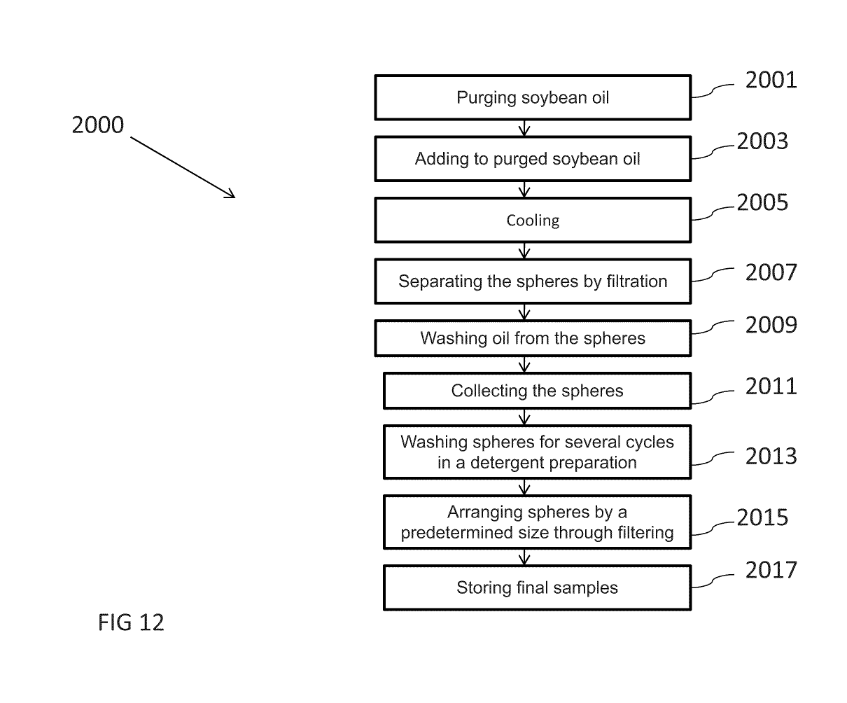

Referring to FIG. 12, in other embodiments PEVTS is used (process 2000) to mechanically process the preform into the three-dimensional microfiber extrudate structure by exposing the preform to external energy such as heat, ultrasound, or a combination thereof. In one embodiment, the polymer in the matrix is brought to a visco-elastic state of relaxation in a suitable medium. The PEVTS can be based on the melt-flow visco-elastic dynamics of a solid polar biodegradable or biocompatible polymer/active matrix composition suspended within a non-polar immiscible liquid.

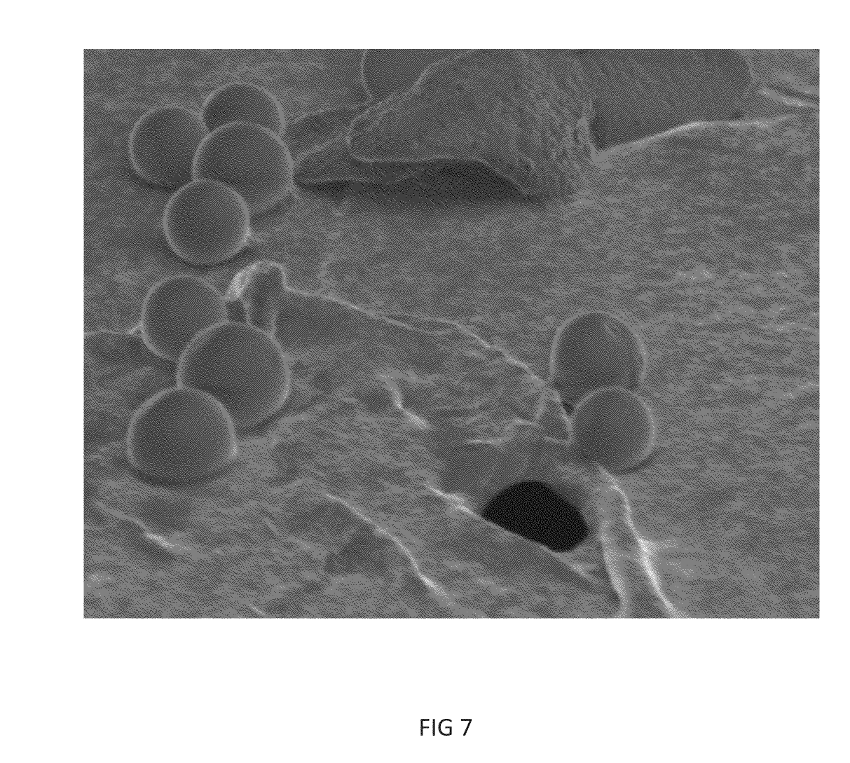

In one embodiment, soybean oil is used as the medium. The soybean oil can be stabilized against thermal oxidation (for example, with 100 ppm of DL-α-tocopherol). The soybean oil and/or other components involved in remodeling and/or washing can be additionally purged with nitrogen (step 2001) to remove free oxygen. The preform can be introduced to the soybean oil (step 2003). In one embodiment, to avoid agglomeration, the introduction of the preform may be performed slowly and under mixing power that is continuous and low-power at room temperature until the mass is uniformly dispersed. For example, oil can be slowly heated for a predetermined period (for example, about 2 hours) while stirring to a predetermined temperature (for example, about 150° C.), then cooled to a second predetermined temperature (for example, room temperature) before handling. In one embodiment, as the temperature of oil increases, heat can be transferred to the preform. As the temperature of the oil approaches a predetermined temperature at which visco-elastic behavior occurs, the preform can remodel to be arranged into a sphere to accommodate the thermal burden. The temperature at which visco-elastic behavior occurs may depend upon composition and/or the use of external sources, such as exposure to ultrasonic or infrared energy. As shown in FIG. 7, this transformation can be similar to blowing soap bubbles. The geometry is cylindrically oblongated until the management of physical and chemical forces on the particle take a spherical shape at the thermal equilibrium point.

When the polymer reaches a visco-elastic relaxation state in the warm oil medium it can assume a low energy shape, which is generally three-dimensional microfiber extrudate structure such as a sphere. The medium can then be cooled (step 2005) and the three-dimensional microfiber extrudate structures can be separated by filtration (step 2007). In one embodiment, the oil can be washed from the three-dimensional microfiber extrudate structures (step 2009) with a detergent formulation including components which are approved for intravenous injection. Three-dimensional microfiber extrudate structures can then be collected (step 2011). Collection can be, for example, in a dutch-twill screen having a predetermined screen size (for example, about 5 μm). Three-dimensional microfiber extrudate structures can then be washed for several cycles in a detergent preparation (step 2013). The detergent preparation can include, for example, 100 parts of water, 0.2 parts soy lecithin, and 0.025 parts glycerin. In one embodiment, the detergent preparation is prepared by dissolving glycerin in water, then adding lecithin. Final collection of three-dimensional microfiber extrudate structures from the wash can be arranged by a predetermined size through filtering (step 2015). For example, 20 μm dutch-twill and 5 μm dutch-twill screens can be used to collect spheres in the range of between about 5 μm up to about 25 μm, or about 10 μm to about 25 μm. Final three-dimensional microfiber extrudate structures can then be stored (step 2017). In one embodiment, the final three-dimensional microfiber extrudate structures are stored in soybean oil. Generally, any residual soybean oil listed on the FDA Inactive Ingredients Guide (IIG) as approved for intravenous injection can be used as an FDA compliant material. Other FDA compliant substances listed on the FDA Inactive Ingredients Guide may be used instead of soybean oil. It will further be appreciated that sphericalized and other shaped particles made in accordance with the PEVTS method described herein could be processed and/or stored in other non-biocompatible fluids, depending on the particular end use for which the particles will be used.

FIGS. 3, 8, and 9 show cross-sections of exemplary three-dimensional microfiber extrudate structures 300. In the embodiments, the three-dimensional microfiber extrudate structure 300 is formed and designed to arrange discrete domains 304, 306 of different materials or combinations of materials, such as an exogenously excitable material and/or an active load within a matrix 302. Each domain can harbor a preferred chemistry for a specific action. Each domain may include the exogenously excitable material, the active load, or a combination of them or other materials. Each domain may also include a certain percent of matrix material to facilitate excitement or to prevent excitement. The number and location of discrete domains of different materials is exemplary and may be modified depending upon the application.

Referring to FIG. 3, the three-dimensional microfiber extrudate structure 300 can include a bio-compatible polymer matrix 302 and a first discrete domain 304 at the core that may contain a suitable bio-active material that may be selected depending upon the desired therapy. As shown in FIGS. 3, 8, and 9, the arrangement of discrete domains 304, 306 and/or polymer matrix 302 can be varied, as further described with reference to the preform in U.S. patent application Ser. No. 12/342,830.

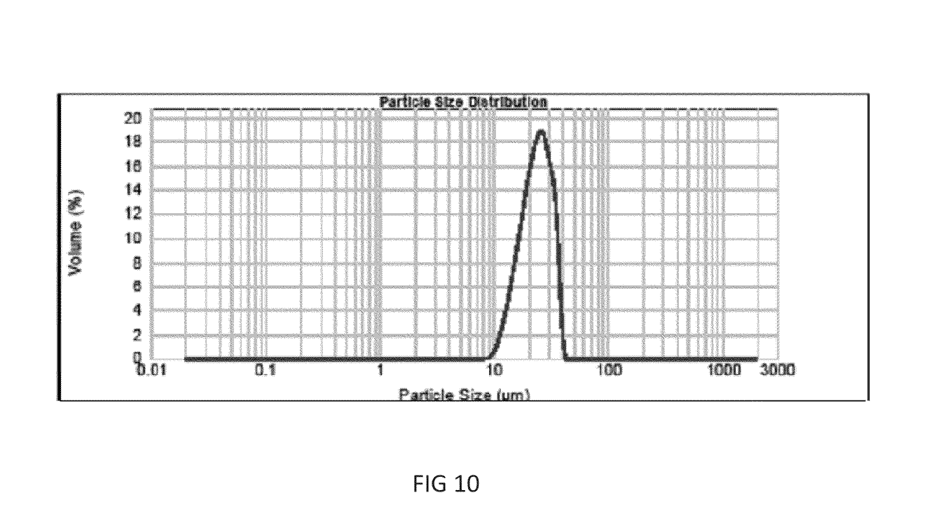

The three-dimensional microfiber extrudate structure may be constructed to include discrete domains with approved excipient materials that contain API or a combination of API and inactive or functional domains within the three-dimensional microfiber extrudate structure. Outside of the domains, the three-dimensional microfiber extrudate structure may additionally or alternatively include approved excipient materials which contain API, inactive materials or functional materials, or a combination of API and inactive or functional materials. The three-dimensional microfiber extrudate structure can be designed to have a wide range of sizes (for example, about 5 μm, about 10 μm, about 40 μm, about 300 μm, about 100 nm) or a distribution of sizes (for example, between about 10 μm and about 50 μm, between about 5 μm and about 100 nm, between about 30 μm and about 50 μm) with a corresponding distribution of sizes (for example, a particle distribution ranging from about 1% of the particles being about 10 μm, about 19% of the particles being about 28 μm, and about 2% of the particles being about 40 μm) or any other suitable distribution. For example, referring to FIG. 10, the three-dimensional microfiber extrudate structure may have a volume differing based upon the particle size. Consequently, a self-contained drug delivery device in accordance with exemplary embodiments in the size range of circulatory cells can be provided and medically administered intravenously or parenternally.

The API, which may be the active load, may be any therapeutic material. Active pharmaceutical ingredients may include, but are not limited to, ABVD, AVICINE, Acetaminophen, Acridine carboxamide, Actinomycin, Alkylating antineoplastic agent, 17-N-Allylamino-17-demethoxygeldanamycin, Aminopterin, Amsacrine, Anthracycline, Antineoplastic, Antineoplaston, Antitumorigenic herbs, 5-Azacytidine, Azathioprine, BBR3464, BL22, Biosynthesis of doxorubicin, Biricodar, Bleomycin, Bortezomib, Bryostatin, Busulfan, Calyculin, Camptothecin, Capecitabine, Carboplatin, Chlorambucil, Cisplatin, Cladribine, Clofarabine, Cyclophosphamide, Cytarabine, Dacarbazine, Dasatinib, Daunorubicin, Decitabine, Dichloroacetic acid, Discodermolide, Docetaxel, Doxorubicin, Epirubicin, Epothilone, Estramustine, Etoposide, Exatecan, Exisulind, Ferruginol, Floxuridine, Fludarabine, Fluorouracil, 5-Fluorouricil, Fosfestrol, Fotemustine, Gemcitabine, Hydroxyurea, Idarubicin, Ifosfamide, Imiquimod, Irinotecan, Irofulven, Ixabepilone, Lapatinib, Lenalidomide, Liposomal daunorubicin, Lurtotecan, Mafosfamide, Masoprocol, Mechlorethamine, Melphalan, Mercaptopurine, Methotrexate, Mitomycin, Mitotane, Mitoxantrone, Nelarabine, Nilotinib, Nitrogen mustard, Oxaliplatin, PAC-1, Paclitaxel, Pawpaw, Pemetrexed, Pentostatin, Pipobroman, Pixantrone, Polyaspirin, Plicamycin, Procarbazine, Proteasome inhibitor, Raltitrexed, Rebeccamycin, SN-38, Salinosporamide A, Satraplatin, Stanford V, Streptozotocin, Swainsonine, Taxane, Tegafur-uracil, Temozolomide, ThioTEPA, Tioguanine, Topotecan, Trabectedin, Tretinoin, Tris(2-chloroethyl)amine, Troxacitabine, Uracil mustard, Valrubicin, Vinblastine, Vincristine, Vinorelbine, Vorinostat, Zosuquidar, and combinations thereof.

The concentration of API (or other active agent) can be at about the maximum concentration that a preselected polymer can sustain while retaining desired melt-flow characteristics. The maximum concentration can vary according to the chemistry of the API and a biopolymer. Generally, soybean oil acts only as a medium for the sphericalization process and does not penetrate PLGA. Table 1 shows commonly used active agents and additional properties of the agents (for example, solubility and thermal stability) that may be considered in making the three-dimensional microfiber extrudate structure.

TABLE 1

| Agent | Classification | Uses | Solubility | Thermal Stability | Matrix |
|---|---|---|---|---|---|
| 5-Fluorouracil | Antimetabolite | Breast, colon, | Soluble in high | Melts 282-283° C. | PLA, PLGA, |
| | | rectal, | pH H₂O, | with | Ethocel, PCL |
| | | pancreatic, | slightly soluble | decomposition | |
| | | stomach | in H₂O, DMF | | |
| | | cancer | | | |
| Altretamine | Alkylating agent | Ovarian | slightly soluble | Tₘ 172-174° C. | PCL |
| | | cancer | in H₂O | | |
| Carboplatin | Alkylating agent | Ovarian, lung, | Sparingly | Melts ~200° C. | PLA, PLGA, |
| | | head and neck | soluble in H₂O, | with | Ethocel, PCL |
| | | cancer | very slightly | decomposition | |
| | | | soluble in | | |
| | | | acetone and | | |
| | | | alcohol | | |
| Cisplatin | Alkylating agent | Bladder, | slightly soluble | Tₘ 270° C. | PLA, PLGA, |
| | | ovarian, | in H₂O | | Ethocel, PCL |
| | | testicular | | | |
| | | cancer | | | |
| Docetaxel | | Breast, lung, | Practically | Tₘ 232° C. | PLA, PLGA, |
| | | prostate | insoluble in | | Ethocel, PCL |
| | | cancer | H₂O | | |
| Doxorubicin | Anthracycline | Broad based | Soluble in H₂O | Tₘ 204-205° C. | PLA, PLGA, |
| | antibiotic | anti- | | | Ethocel, PCL |
| | | neoplastic | | | |
| Letrozole | aromatase | Breast cancer | insoluble in | Tₘ 184-185° C. | PLA, PLGA, |
| | inhibitor | | H₂O | | Ethocel, PCL |
| Leucovorin | Synergist, side | Adjuvant used | Soluble in H₂O | Tₘ 240-250° C. | PLA, PLGA, |
| | effect reducer | with 5-FU or | | | Ethocel, PCL |
| | | Methotrexate | | | |
| Melphalan | | Multiple | Sparingly | Tₘ 182.5° C. | PLA, PLGA, |
| | | myeloma, | soluble in H₂O | | Ethocel, PCL |
| | | ovarian | | | |
| | | cancer | | | |
| Methotrexate | antimetabolite | Broad based | slightly soluble | Tₘ 185-195° C. | PLA, PLGA, |
| | | anti- | in H₂O | with | Ethocel, PCL |
| | | neoplastic | | decomposition | |
| Mitomycin | Anti-tumor | Broad based | slightly soluble | Tₘ >360° C. | PLA, PLGA, |
| | antibiotic | anti- | in H₂O | | Ethocel, PCL |
| | | neoplastic | | | |
| Paclitaxel | Mitotic inhibitor | Broad based | insoluble in | Tₘ 213-216° C. | PLA, PLGA, |
| | | anti- | H₂O | | Ethocel, PCL |
| | | neoplastic | | | |
| Pentostatin | | leukemia | slightly soluble | Tₘ 220° C. | PLA, PLGA, |
| | | | in H₂O | | Ethocel, PCL |
| Tamoxifen | Anti-estrogen | Breast cancer | slightly soluble | Tₘ 97° C. | PCL |
| | | | in H₂O | | |

One example of a liposome approved for intravenous drug delivery is Doxil®, a liposomal form of doxorubicin. In the manufacture of liposomes, it is necessary to dissolve the drug in either the hydrophilic core or the hydrophobic bi-layer membrane of the liposome, thereby narrowing the choices of drugs which can be incorporated at significant concentration. Liposomes can seep through endothelial fenestrations in tumor vasculature to attack tumor cells, but can also target capillaries of the hands and feet resulting in unwanted side effects such as Palmer-Planter Erythrodysesthesia. Liposomes can also be rendered ineffective by heterogeneous interstitial pressure gradients inherent with aggressive tumors, resulting in liposome clusters just outside the endothelial wall. Drugs released as small molecules, such as those released from microspheres, have a higher likelihood of penetrating the interstitial microenvironment to reach aggressively multiplying tumor cells.

In one embodiment, the API may be matched by decomposition point to the melt-flow temperature of the biopolymer of choice and can be extruded as a solid powder or in the melt. In one embodiment, polymers can be custom plasticized to modify melt-flow to lower temperatures than what is reported for neat polymers. This can aid in processing temperature sensitive API. In one embodiment, the polymer melt-flow temperature may be at least 25-50° C. below the decomposition point of the API. In this embodiment, anti-oxidation or thermal stabilization may be achieved with tocopherol (which is a traditional phenolic anti-oxidant). In other embodiments, excipients such as citric acid or benzoic acid with known anti-oxidant behavior can be employed for stabilization in extrusion.

The three-dimensional microfiber extrudate structure can include drugs are insoluble in water and therefore have limited usefulness in traditional drug delivery methods. In order for an insoluble drug to be delivered via the microfiber extrudate, it can first be incorporated into a biopolymer via extrusion with consideration for the degradation temperature of the insoluble drug. The degradation temperature of the insoluble drug can be higher than the extrusion temperature of the biopolymer in which the drug is being incorporated to avoid the risk the drug may degraded during the extrusion process.

A variety of biopolymers are available that can be processed via extrusion processes discussed herein. The extrusion processing of biopolymers typically range from temperatures of 140° C. to 260° C., but may be as low as 60° C. or lower, for example, depending upon composition, including any plasticizers which may be employed. Table 2 shows processing temperatures for several common biodegradable polymers in light of melting and decomposition temperatures of those materials.

TABLE 2

| Polymer | Processing Temperature |
|---|---|
| 100% Poly glycolic acid | 240-260° C |
| 100% Poly l-lactic acid | 190-210° C |
| 100% Poly d-lactic acid | 191-170° C |
| 90%/10% glycolide co-l-lactide | 200-220° C |
| 70%/30% l-lactide/polycaprolactone | 201-170° C |
| 50%/50% d,l-lactide co-glycolide | 140-170° C |

Just as in conventional thermoplastic processing, biopolymers have “windows” in which they can be processed in order to create the finished item of interest (i.e. fiber, film, sphere, rod, disk, etc.). This processing window can be based upon the individual polymer's glass transition temperature (in the case of amorphous polymers), melt temperature (in the case of crystalline polymers) and degradation temperature. The insoluble drug incorporated into the biopolymer can be thermally stable up to the processing temperature of that biopolymer.

Other therapeutic materials such as anti-tumor antibodies (including VEGH-A or other monoclonal antibodies, for example), antibiotics, bio-agents, bio-pharmaceuticals and/or other suitable therapeutic materials may be included. Additionally or alternatively, diagnostic materials, matrix diffusion control materials, and/or other suitable materials may be included.

If an exogenously excitable material is included, it may be selected as any material capable of being excited by an exogenous stimulus. The exogenous stimuli include, but are not limited to, radiofrequency excitation, microwave excitation, terahertz excitation, mid infrared excitation, near infrared excitation, visible excitation, ultraviolet excitation, x-irradiation excitation, magnetic excitation, electron beam irradiation excitation, and combinations thereof. Upon receiving the exogenous stimulus, the exogenously excitable material can be excited. The exogenously excitable material may be arranged within the domains in the three-dimensional microfiber extrudate structure or may be mixed within the matrix. Various therapies may combine exogenously excitable materials in the three-dimensional microfiber extrudate structure along with the API.

In one embodiment, the three-dimensional microfiber extrudate structure may include a sensitive additive (for example, a radiofrequency (RF) sensitive additive) as the exogenously excitable material and a degradable polymer as a bio-compatible matrix that can be administered. The exogenously excitable material may be exogenously excited in situ at the local site of tumor angiogenesis, such as a receptor specific region in advancing vascular tissue binding VEGF to facilitate localized heating and thereby denaturing angiogenesis factors and/or destroying abnormal cells at the advancing site. Where the API is the active load, the excitation may be configured to expedite breakdown of the matrix, thus releasing the pharmaceutical more quickly. In RF active embodiments, the microfiber matrix may be formulated with a known additive having a known radiofrequency, lambda max or excitation frequency, which can then be exogenously excited. In another approach, the natural RF response of the extrudate in the absence of a specific radiosensitive additive is determined by some diagnostic mechanism like MRI, and a tunable RF generator may be used to administer the exogenous non-ionizing radiation. As will be appreciated by those skilled in the art, other exogenously excitable materials may be similarly utilized.

While the disclosure has been described with reference to a preferred embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

What is claimed is:

1. A process for forming a three-dimensional microfiber extrudate structure, the process comprising:
introducing a preform to a medium;
maintaining the preform in the medium at least until a visco-elastic relaxation state is reached;
wherein the three-dimensional microfiber extrudate structure is formed by the preform reaching the visco-elastic relaxation state.

2. The process of claim 1, further comprising drying the three-dimensional microfiber extrudate structure.

3. The process of claim 2, wherein a geometry of the three-dimensional microfiber extrudate is substantially maintained after drying.

4. The process of claim 1, wherein the three-dimensional microfiber extrudate structure consists essentially of materials that are safe for humans.

5. The process of claim 1, wherein the three-dimensional geometry is deformable.

6. The process of claim 1, wherein the preform includes a solid polar biodegradable matrix and the medium includes a non-polar immiscible liquid.

7. The process of claim 1, wherein the three-dimensional microfiber extrudate structure includes an active pharmaceutical ingredient.

8. The process of claim 1, wherein the preform includes a biocompatible matrix and the medium includes a non-polar immiscible liquid.

9. The process of claim 1, wherein the medium includes soybean oil.

10. The process of claim 1, wherein the medium is stabilized against thermal oxidation with DL-a-tocopherol.

11. The process of claim 1, wherein the three-dimensional microfiber extrudate structure is spherical.

12. The process of claim 1, further comprising cooling the three-dimensional microfiber extrudate structure.

13. The process of claim 1, further comprising filtering and collecting the three-dimensional microfiber extrudate structure.

14. The process of claim 1, further comprising storing the three-dimensional microfiber extrudate in the medium.

15. The process of claim 1, wherein the three-dimensional microfiber extrudate structure includes otherwise incompatible components.

16. The process of claim 1, wherein the preform includes a polymer, the polymer including an amorphous drug formed by dispersing a crystalline drug in the polymer.

17. A three-dimensional microfiber extrudate structure, comprising:
a matrix having a three-dimensional geometry;
wherein the three-dimensional geometry is a visco-elastic relaxation state of a preform introduced to a medium;
wherein the three-dimensional geometry is deformable.

18. The three-dimensional microfiber extrudate structure of claim 15, wherein the three-dimensional geometry is capable of being substantially maintained after drying of the three-dimensional microfiber extrudate structure.

19. A three-dimensional microfiber extrudate structure, comprising:
a matrix having a three-dimensional geometry;
wherein the three-dimensional geometry is a visco-elastic relaxation state of a preform introduced to a medium;
wherein the matrix consists essentially of materials that are safe for humans.

20. The three-dimensional microfiber extrudate structure of claim 18, wherein the three-dimensional geometry is capable of being substantially maintained after drying of the three-dimensional microfiber extrudate structure.
